(19) United States
(12) Patent Application Publication
(10) Pub. No.: US 2024/0066164 A1
(43) Pub. Date: Feb. 29, 2024

(54) ISOLATOR FOR PROCESSING MEDICAL SUBSTANCES AND METHOD FOR DECONTAMINATING AN ISOLATOR

(71) Applicant: Syntegon Technology GmbH, Waiblingen (DE)

(72) Inventors: Benjamin Hoffmann, Vellberg (DE); Albrecht Kühnle, Crailsheim (DE); Thomas Kosian, Insingen (DE)

(21) Appl. No.: 18/261,142

(22) PCT Filed: Dec. 15, 2021
(86) PCT No.: PCT/EP2021/085813

§ 371 Date: Jul. 12, 2023

(30) Foreign Application Priority Data
Jan. 22, 2021 (DE) 10 2021 101 402.6

(51) Int. Cl. A61L 2/20; A61L 2/26; B01L 1/02; F24F 3/167

(52) U.S. Cl. CPC A61L 2/208; A61L 2/26; B01L 1/025; F24F 3/167; A61L 2202/11; A61L 2202/122; A61L 2202/123; A61L 2202/14

(57) ABSTRACT

The invention relates to an isolator (10) for processing medical substances and a method for decontamination of an isolator (10).

### BACKGROUND

The invention relates to an isolator for processing medical substances, and to a method for decontamination of an isolator.

Isolators for processing or filling medical agents under sterile conditions are generally known. Isolators must be decontaminated regularly. This is usually done by introducing a hydrogen peroxide (H2O2)—/air mixture into the isolator.

After decontamination, it is important to flush the isolator to remove hydrogen peroxide residues. The flushing is usually carried out with air.

Flushing of the isolator can be realized e.g., via recirculated air lines (see below) or with the aid of catalysts in the recirculated air.

For example, DE 10 2014 202 592 A1 discloses such an isolator with a corresponding decontamination process.

Within the isolator or across the isolator boundaries, there may be further supply or discharge lines in addition to recirculated air lines. Recirculated air lines means lines through which the recirculated air of the isolator is conducted. By recirculated air is meant air from the isolator that is recirculated. The further supply or discharge lines just mentioned are often very thin or of small cross-section. Such a discharge line from the isolator can be, for example, a particle measuring line via which a sample of the recirculated air can be extracted from the isolator. A supply line can be, for example, a working gas supply line for a working gas supply during production. The working gas can be, for example, compressed air for the pneumatics of a machine inside the isolator and used for its operation. The working gas can be nitrogen, which can be used, for example, to reduce the oxygen content of a container located inside the isolator. These supply and discharge lines must also be decontaminated and then flushed.

Usually, the supply and discharge lines, which have a small cross-section, are decontaminated by suctioning the H2O2 mixture from inside the isolator through the supply and discharge lines and reintroducing it into the isolator. Such suction via the supply and discharge lines and reintroduction of the H2O2 mixture into the isolator is also referred to as a (bio)decontamination loop. A disadvantage here is the long time required for such a decontamination process. It takes a certain time to build up an effective H2O2-air mixture inside the Isolator. Moreover, additional pumps must be connected to the supply and discharge lines in order to extract the H2O2 mixture from inside the isolator.

### SUMMARY

The task of the present invention is to provide an isolator for processing medical substances and a method for decontamination of an isolator, which eliminate the above disadvantages.

The isolator comprises a working space sealed off from its environment and at least one supply line. Alternatively or in addition thereto, the isolator may comprise a discharge line. In particular, the isolator may comprise a supply line and a discharge line. In particular, the isolator may comprise exactly one supply line and exactly one discharge line.

The supply line can supply a gas to a machine component located inside the working space.

The discharge line can discharge a gas from a machine component located inside the working space.

Machine components can be, for example, a pneumatically actuated gripper or robot arm. But also hoses, tubes, valves, filters, needles are to be understood as machine components in the sense of the present application.

The supply line is in fluidic connection with the working space. Alternatively or additionally, the discharge line is in fluidic connection with the working space. In other words, a fluid/gas can flow between the supply and/or discharge line and the working space.

A fluidic connection or fluidic coupling means that a gas (fluid) can flow between two fluidically coupled elements or between two elements in fluidic connection.

The isolator further comprises a decontamination device for decontaminating the isolator. This is done by introducing a decontaminant. The decontamination device has at least one first decontamination line for introducing the decontaminant.

The decontaminant can be in gaseous form. However, it is also conceivable that the decontaminant is liquid or partially liquid. In particular, the decontaminant is hydrogen peroxide (H2O2). In particular, the decontaminant is gaseous hydrogen peroxide (H2O2), especially an H2O2/air mixture. For this purpose, a solution of e.g., 35% is evaporated and introduced into the isolator to be decontaminated.

The decontamination device is designed to supply the decontaminant to the working space of the isolator via the first decontamination line and at least partially via the supply and/or discharge line.

The decontaminant can thus be supplied to the working space of the isolator via the decontamination line and at least partially via the supply line.

Alternatively or additionally, the decontaminant can be supplied to the working space of the isolator via the decontamination line and at least partially via the discharge line.

Due to the fact that the decontaminant is at least partially supplied to the working space via the supply and/or discharge line, it is possible that (temporarily) contaminated material from the supply and/or discharge line may be introduced into the working space of the isolator. However, this is not considered critical due to the rapid build-up of the H2O2 concentration. In addition, it is conceivable that a corresponding filter can be provided in the supply and/or discharge line, which filters contaminated material from the supply and/or discharge line and thus prevents contaminated material from being fed from the supply and/or discharge line into the working space.

This means that an H2O2 mixture is no longer extracted from the work space through the supply line or the discharge line during decontamination, as is usually the case. Instead, the decontaminant from the first decontamination line now flows directly through the supply line or the discharge line. This results in a much faster build-up of the critical H2O2 concentration in the supply and/or discharge line. Thus, a faster/efficient decontamination effect in the (bio-) decontamination loop can be assumed, since the decontaminant is no longer sucked in diluted from the working space of the isolator via the supply line and/or the discharge line (it takes a certain time until a high concentration of the decontaminant is present there). Furthermore, additional pumps can be omitted, in particular pumps of the supply and/or discharge line are no longer flown through with H2O2.

The supply line may be a working gas supply line for an additional gas supply during an operation of the isolator. The gas supply can be a working gas. The working gas may be, for example, compressed air that can be used to pneumatically control a machine. For example, machine components within the working space can be pneumatically actuated. Likewise, it is conceivable that the working gas can be used, for example, as a protective gas for processes within the working space. It is also conceivable that compressed air, another fluid or a combination of compressed air and another fluid is supplied via the supply line.

The discharge line can be a particle measuring line. The particle concentration in the recirculated air in the working space can be monitored via a particle measuring line. For this purpose, a sample of the recirculated air can be extracted from the working space via the particle measuring line and fed to a particle measuring device, for example. In other words, air is extracted from the working space via the particle measurement line and subjected to a particle measurement, in particular by means of a particle measurement device. This can be used to monitor the working space atmosphere (particle concentration in the working space), in particular during an operation of the isolator. The particle measuring device can be designed as part of the isolator.

The discharge line can be designed as a vacuum line. A vacuum can therefore be introduced into the working space via the discharge line or vacuum line, e.g., applied to a machine component. For example, a vacuum gripper (machine component) can be supplied with a corresponding vacuum

The decontamination device can have at least one second decontamination line for introducing the decontaminant. The decontamination device can be designed to supply the decontaminant to the working space of the isolator via the second decontamination line.

The decontaminant can be introduced into the working space via the first decontamination line and at least partially via the supply line and at the same time, with a time delay or at different time intervals via the second decontamination line.

Alternatively or additionally, the decontaminant can be introduced into the working space via the first decontamination line and at least partially via the discharge line and, in the doing, at the same time, with a time delay or at different time intervals via the second decontamination line.

The first and second decontamination lines can be designed separately from each other as separate lines. However, it is also conceivable that the first and second decontamination lines are designed as one (common) line, at least in sections.

In particular, the first decontamination line may run between the decontamination device and the supply line and, alternatively or additionally, between the decontamination device and the discharge line. In particular, the second decontamination line may run between the decontamination device and the working space of the isolator.

In particular, however, it is also conceivable that a common line section of the first and second decontamination lines is arranged on the decontamination device. The first decontamination line can run between this common line section and the working space. In this regard, the second decontamination line may extend between this common line section and the supply line. Alternatively or additionally, the second decontamination line may run between this common line section and the discharge line.

The isolator can have a flushing device for removing the decontaminant. This can be designed to supply flushing medium via the first decontamination line and at least partially via the supply line into the working space of the isolator. Alternatively or in addition thereto, the flushing device may be designed to supply flushing medium via the first decontamination line and at least partially via the discharge line into the working space of the isolator. Alternatively or additionally, the flushing device can be designed to supply flushing medium via the second decontamination line into the working space of the isolator. The flushing medium can be compressed air.

The decontamination device can have an evaporator. This can be designed to convert the decontaminant from a liquid phase and/or solid phase into a gaseous phase by evaporation.

It is conceivable that the evaporator is arranged inside the working space of the isolator. However, the evaporator can also be arranged outside the working space of the isolator.

It is also conceivable that the decontaminant can be introduced into the decontamination device already in gaseous form and in a desired concentration.

The decontamination device can have a compressed air supply. This can be fluidically coupled to the evaporator. Alternatively or in addition thereto, the compressed air supply may be fluidically coupled to the first decontamination line. Alternatively or additionally thereto, the compressed air supply may be fluidically coupled to the second decontamination line. The compressed air supply can be used for flushing (compressed air as flushing medium) the respective lines or elements. The compressed air supply can also be used as a carrier or transport medium for the decontaminant. The compressed air supply can be used to dilute/achieve the desired concentration of the decontaminant. In particular, the compressed air supply may be part of the flushing device, or constitute the flushing device.

The task to be solved is further solved by a method for decontamination of an isolator. In this case, the isolator comprises a working space which is sealed off from its environment. The isolator comprises at least one supply line for supplying a gas, in particular to at least one machine component arranged within the working space. Alternatively or additionally thereto, the isolator comprises a discharge line for discharging a gas, in particular from at least one machine component arranged within the working space. In this case, the supply line is fluidically coupled to the working space. Alternatively or additionally, the discharge line is fluidically coupled to the working space.

The method comprises the step:

Introducing a decontaminant into the working space of the isolator via at least one first decontamination line and at least partially via the supply line. Alternatively or in addition thereto, the decontaminant can be introduced into the working space of the isolator via at least one first decontamination line and at least partially via the supply line.

The decontaminant can be gaseous. However, it is also conceivable that the decontaminant can be liquid. In particular, the decontaminant is hydrogen peroxide (H2O2). In particular, the decontaminant is gaseous hydrogen peroxide (H2O2), especially an H2O2/air mixture.

This means that the H2O2 mixture is no longer extracted from the work space through the supply line or the discharge line during decontamination, as is traditionally the case. Instead, the decontaminant now flows directly through the supply line or the discharge line. For this purpose, compressed air and H2O2 can be applied to the supply line or the discharge line. This results in a much faster build-up of the critical H2O2 concentration in the supply line or in the discharge line. Thus, a faster/efficient decontamination effect in the (bio-) decontamination loop can be assumed, since the decontaminant is no longer sucked in diluted from the working space of the isolator via the supply line or discharge line (it takes a certain time until a high concentration of the decontaminant is present there). In addition, pumping/suction, in particular by means of additional pumps, can be dispensed with, and in particular the H2O2 no longer flows through them.

The method may further comprise the step of:

Introducing the decontaminant into the working space via a second decontamination line.

The decontaminant can be introduced into the working space via the first decontamination line and at least partially via the supply line and at the same time, with a time delay or at different time intervals via the second decontamination line

Alternatively or additionally, the decontaminant can be introduced into the working space via the first decontamination line and at least partially via the discharge line and at the same time, with a time delay or at different time intervals via the second decontamination line.

The method may further comprise the step of:

Converting the decontaminant from a liquid phase to a gaseous phase. It is also conceivable to convert the decontaminant from a solid phase to a gaseous phase. This can be realized by means of evaporation, in particular by means of an evaporator.

The method may further comprise the step of:

Pressurizing the decontaminant with compressed air. A compressed air supply may be provided for this purpose.

The method may further comprise the step of:

Introducing a flushing medium via the first decontamination line and at least partially via the supply line into the working space of the isolator. Alternatively or additionally thereto, the flushing medium may be introduced via the first decontamination line and at least partially via the discharge line into the working space of the isolator. Alternatively or additionally, the flushing medium can be introduced into the working space of the isolator via the second decontamination line.

The flushing medium can be compressed air, in particular, which is introduced by means of a compressed air supply.

Removal of the H2O2 by flushing with compressed air according to the procedure described above is particularly efficient because the compressed air (flushing medium) is not contaminated with H2O2. This would be the flushing medium if it were pumped out of the working space through the supply and/or discharge lines. In addition, much higher air volumes/speeds can be realized with compressed air. Otherwise, even larger/more expensive vacuum pumps would have to be used specifically for flushing.

An isolator with the features described above can be used to execute the method.

### BRIEF DESCRIPTION OF THE DRAWING

Further features, details and advantages of the invention are apparent from the disclosure and from the following description of the embodiment example based on the drawing. It shows:

FIG. 1 a schematic illustration of an isolator according to the invention.

### DETAILED DESCRIPTION

In the following description and in the FIG., the corresponding components and elements have the same reference signs.

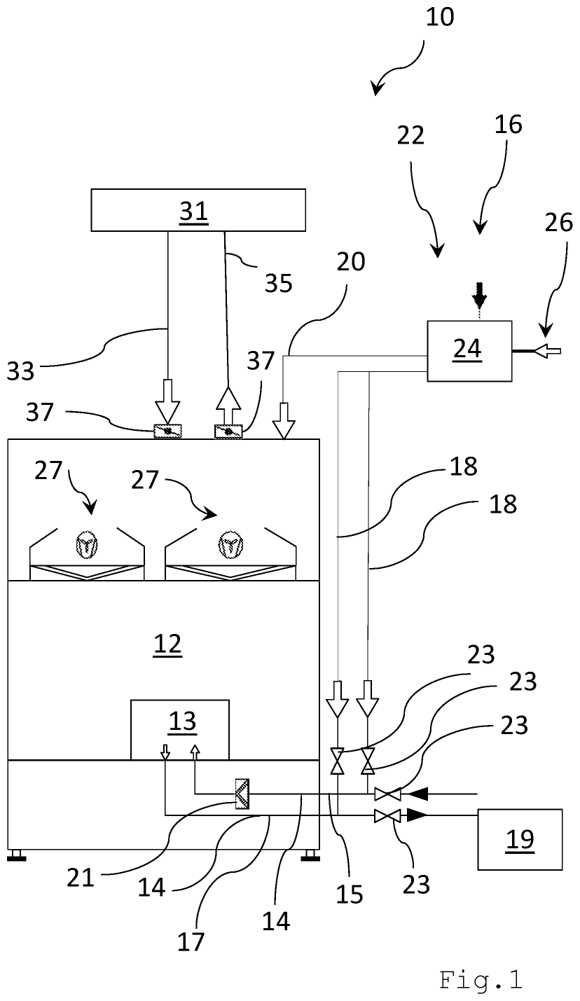

FIG. 1 shows a schematic illustration of an isolator 10 according to the invention. The isolator 10 comprises a working space 12 which is sealed off from its environment.

At least one machine component 13 is arranged within the working space 12. The isolator 10 has at least one supply and/or discharge line 14. In the present case, the isolator 10 has an supply and an discharge line 14. The supply and a discharge line 14 are fluidically coupled to the working space 12.

In the present case, the supply line 14 is formed as a working gas supply line 15 for a working gas during an operation of the isolator 10. The working gas may be in the form of compressed air for a pneumatic machine. Material (e.g., in the form of gas and/or foreign particles) may enter the isolator 10 through the working gas supply line 15. Therefore, a filter 21 is arranged on the working gas supply line 15 to prevent unwanted material from entering the working space 12 of the isolator 10 through the working gas supply line 15.

In the present case, the discharge line 14 is designed as a particle measuring line 17. The particle measuring line 17 can be used to take an air sample from the working space 12 and to determine the particles present in the air sample. By means of such a particle sampling from the working space 12, a monitoring of the working space atmosphere can be realized by means of a particle measurement. A particle measuring device 19 is provided for this purpose, into which the particle measuring line 17 leads. The concentration of the measured particles in the working space atmosphere can be determined by the particle measuring device 19. It is also conceivable that the particle measuring device 19 identifies the particles. In this way, for example, it can be concluded that certain (undesirable) substances are present in the working atmosphere of the working space 12.

The flow directions in the working gas supply line 15 and the particle measurement line 17 during operation of the isolator 10 are indicated by arrows and black triangles, respectively

In the present case, the working gas supply line 15 and the particle measuring line 17 can each be fluidically decoupled from their environment by a valve 23.

The isolator 10 has two recirculated air filters 27 in its working space 12, which filter the recirculated air within the working space 12. The recirculated air is introduced to the working space 12 by means of a ventilation device 31 through a recirculated air supply 33. The direction of flow of the recirculated air supply 33 is indicated by an arrow in the direction of the working space 12.

The recirculated air from the working space 12 is discharged in the form of exhaust air via the recirculated air discharge 35 by means of the ventilation device 31. Here, too, the direction of flow is indicated by an arrow (pointing away from the working space 12).

In the present case, the recirculated air supply 33 and the recirculated air discharge 35 can each be fluidically disconnected by means of a shut-off flap 37. It is conceivable that by means of the shut-off flap 37 the volume flow (flow rate) of the air through the recirculated air supply 33 or the recirculated air discharge 35 can be regulated. It is also conceivable that valves can be used instead of shut-off flaps 37.

It is conceivable that the recirculated air supply 33 and/or the recirculated air discharge 35 can be fluidically coupled to the environment of the isolator 10 at least in part, in particular via the ventilation device 31. For example, fresh air from the environment of the isolator 10 can be supplied to the working space 12 of the isolator 10 via the recirculated air supply 33. Correspondingly, exhaust air from the working space 12 of the isolator 10 can be discharged into the environment of the isolator 10 via the recirculated air discharge 35.

The recirculated air supply 33 and the recirculated air discharge 35 are each separate lines from the working gas supply line 15 and particle measuring line 17.

The isolator 10 has a decontamination device 16. The decontamination device 16 is designed to introduce decontaminant into the isolator 10 via decontamination lines.

In the present case, the decontamination device 16 has two first decontamination lines 18. The first of the two first decontamination lines 18 is fluidically coupled to the working gas supply line 15 and the second of the two first decontamination lines 18 is fluidically coupled to the particle measurement line 17.

In the present case, the two first decontamination lines 18 can be fluidically decoupled from the working gas supply line 15 or the particle measuring line 17 by means of the valves 23. The direction of flow of the decontaminant through the first decontamination lines 18 is indicated by an arrow in the direction of the working gas supply line 15 or the particle measuring line 17.

The decontamination device 16 has a second decontamination line 20. This is fluidically coupled to the working space 12 of the isolator 10. The direction of flow of the decontaminant through the second decontamination line 20 is indicated by an arrow in the direction of the working space 12.

The decontamination device 16 has an evaporator 24. This is designed to convert liquid decontaminant into a gaseous phase by evaporation. For example, liquid hydrogen peroxide (H2O2) is supplied to the evaporator 24. This is indicated in FIG. 1 by a black, thick arrow in the direction of the evaporator 24.

The decontamination device 16 further comprises a compressed air supply 26. In the present case, the compressed air supply 26 is designed in such a way that compressed air is introduced into the evaporator 24 (indicated by an arrow in the direction of the evaporator 24). This compressed air mixes with the gaseous hydrogen peroxide (H2O2) evaporated in the evaporator 24. This produces a hydrogen peroxide/air mixture, wherein the concentration of the hydrogen peroxide can be adjusted as desired. The hydrogen peroxide/air mixture thus produced now forms the decontaminant, which is supplied through the first two decontamination lines 18 and the second decontamination line 20.

Thus, on the one hand, the decontaminant is supplied to the working space 12 of the isolator 10 via the second decontamination line 20. On the other hand, the decontaminant is fed via the two second decontamination lines 18 into the working gas supply line 15 and the particle measuring line 17, respectively. The decontaminant then passes through the working gas supply line 15 and the particle measuring line 17, respectively, into the working space 12 of the isolator 10. In this way, the working gas supply line 15 and the particle measuring line 17 are flushed with the decontaminant and decontaminated.

In the present case, the decontaminant concentration introduced into the working space 12 of the isolator 10 by means of the second decontamination line 20 is the same as the decontaminant concentration introduced into the working gas supply line 15 and the particle measurement line 17. This can ensure thorough decontamination of all components of the isolator 10 for which a certain minimum concentration of decontaminant is required.

The isolator 10 also has a flushing device 22. This is designed to introduce flushing medium through the first two decontamination lines 18, the working gas supply line 15 and the particle measurement line 17, respectively, into the working space 12 of the isolator 10. In addition, the flushing device 22 is configured to introduce flushing medium through the decontamination line 20 into the working space 12 of the isolator 10. The flushing medium thereby flushes through the corresponding elements of the isolator 10 and removes the decontaminant (or the residues of the decontaminant). The flushing medium can be compressed air.

In the present case, the flushing device 22 is in the form of the compressed air supply 26. In other words, the compressed air of the compressed air supply 26 can be used to produce a decontaminant mixture with a desired concentration (hydrogen peroxide/air), as a transport medium for the decontaminant and also as a flushing medium for flushing the isolator 10.

1. An isolator (10) for processing medical substances, comprising:
a working space (12) sealed off from the environment,
at least one supply line and/or one discharge line (14) for supplying and/or discharging a gas to and/or from at least one machine component (13) arranged inside the working space (12),
wherein the supply line and/or discharge line (14) is in fluidic communication with the working space (12),
a decontamination device (16) for decontaminating the isolator (10) by introducing a decontaminant,
wherein the decontamination device (16) comprises at least a first decontamination line (18) for introducing the decontaminant,
wherein
the decontamination device (16) is configured to supply at least part of the decontaminant to the working space (12) of the isolator (10) via the first decontamination line (18) and at least partially via the supply line and/or discharge line (14), wherein the machine component (13) is configured as a pneumatically actuated gripper, robot arm, hose, tube, valve or needle.

2. The isolator (10) according to claim 1, wherein the supply line (14) is a working gas supply line (15) for a working gas during an operation of the isolator (10).

3. The isolator (10) according to claim 1, wherein the discharge line (14) is a particle measuring line (17) via which a sample of air in the working space can be drawn off via the particle measuring line (17) and can be fed to a particle measuring device (19).

4. The isolator (10) according to claim 1, wherein the decontamination device (16) for introducing the decontaminant comprises at least a second decontamination line (20) and is configured to supply at least a part of the decontaminant via the second decontamination line (20) into the working space (12) of the isolator (10).

5. The isolator (10) according to claim 4, wherein the isolator (12) comprises a flushing device (22) for removing the decontaminant, which is configured to supply a flushing medium via the first decontamination line (18) and at least partially via the supply and/or discharge line (14) and/or via the second decontamination line (20) into the working space (12) of the isolator (10).

6. The isolator (10) according to claim 1, wherein the decontamination device (16) comprises an evaporator (24) configured to convert the decontaminant by evaporation from a liquid phase and/or solid phase to a gaseous phase.

7. The isolator (10) according to claim 6, wherein the decontamination device (16) comprises a compressed air supply (26) which is in fluidic communication with the evaporator (24), the first decontamination line (18) and/or a second decontamination line (20).

8. A method for decontamination of an isolator (10) with
a working space (12) sealed off from its environment,
at least one supply and/or discharge line (14) for supplying and/or discharging a gas to and/or from at least one machine component (13) arranged inside the working space (12), wherein the machine component (13) is configured as a pneumatically actuated gripper, robot arm, hose, tube, valve or needle, wherein the supply and/or discharge line (14) is in fluidic communication with the working space (12), wherein the method comprises the following steps:
Introducing a decontaminant into the working space (12) of the isolator via at least one first decontamination line (18) and at least partially via the supply and/or discharge line (14).

9. The method according to claim 8, wherein the method further comprises the step of:
Introducing the decontaminant into the working space via a second decontamination line (20).

10. The method according to claim 8, wherein the method further comprises the step of:
Converting the decontaminant from a liquid and/or solid phase to a gaseous phase.

11. The method according to claim 8, wherein the method further comprises the step of:
Pressurizing the decontaminant with compressed air.

12. The method according to claim 9, wherein the method further comprises the step of:
Introducing a flushing medium, via the first decontamination line (18) and at least partially via the supply and/or discharge line (14) and/or via the second decontamination line (20) into the working space (12) of the isolator (10).

13. (canceled)

14. The isolator (10) according to claim 1, wherein the decontaminant is a gaseous decontaminate.

15. The isolator (10) according to claim 1, wherein the decontaminant is hydrogen peroxide.

16. The isolator (1) according to claim 3, wherein the particle measuring device (19) is part of the isolator (10).

17. The isolator (1) according to claim 5, wherein the flushing medium is compressed air.

18. The method according to claim 8, wherein the decontaminant is a gaseous decontaminate.

19. The method according to claim 8, wherein the decontaminant is hydrogen peroxide.

20. The method according to claim 10, wherein converting the decontaminant from a liquid and/or solid phase to a gaseous phase includes using an evaporator (24).

21. The method according to claim 12, wherein the flushing medium is compressed air.
